(19) United States
(12) Patent Application Publication
(10) Pub. No.: US 2023/0149036 A1
(43) Pub. Date: May 18, 2023

(54) Medical Device Having a Single Output and a Dual Output

(71) Applicant: The Regents of the University of Colorado, a body corporate, Denver, CO (US)

(72) Inventors: Maria Albuja Cruz, Denver, CO (US); Brandon Manusov, Friendswood, TX (US)

(21) Appl. No.: 17/991,269

(22) Filed: Nov. 21, 2022

Related U.S. Application Data
(63) Continuation of application No. PCT/US21/33412, filed on May 20, 2021.
(60) Provisional application No. 63/027,446, filed on May 20, 2020.

(51) Int. Cl. A61B 17/28

(52) U.S. Cl. CPC A61B 17/2816; A61B 2017/00367

(57) ABSTRACT

A medical device having a mechanism adapted to convert a single translational input into multiple rotational outputs. An embodiment includes a hinge pin assembly in operable communication with a single input actuator. The hinge pin assembly includes a housing configured to translate in response to actuation of the input actuator. The housing translates about the length of a hinge pin. The housing has a plurality of concentrically aligned annular rings. An upper pinion and a lower pinion are in operable communication with the housing such that linear translation of the housing cause the pinions to rotate. Each pinion is in operable communication with linear gear racks, axles, or other force transferring components. The force transferring components are in operable communication with a pivotable distal end of the medical device. As a result, the linear input causes each distal end of the medical device to pivot with respect to its respective shank.

### CROSS-REFERENCE TO RELATED APPLICATIONS

This nonprovisional application is a continuation of and claims priority to PCT application No. PCT/US21/33412, entitled “A MEDICAL DEVICE HAVING A SINGLE INPUT AND A DUAL OUTPUT,” filed May 20, 2021 by the same inventor(s), which claims priority to provisional application No. 63/027,446, entitled “A MEDICAL DEVICE HAVING A SINGLE INPUT AND A DUAL OUTPUT,” filed May 20, 2020 by the same inventor(s).

### BACKGROUND OF THE INVENTION

### 1. Field of the Invention

This invention relates, generally, to medical equipment. More specifically, it relates to a pair of forceps adapted to convert a single linear input into dual rotation of the distal ends of the forceps.

### 2. Brief Description of the Prior Art

During surgery, surgeons are often forced to exchange a linearly shaped forceps tool as depicted in FIG. 1A for a right-angled forceps tool as depicted in FIG. 1B, or vice versa. Each instrument has its own advantages and disadvantages. Depending on the surgery, a surgeon may be forced to continually exchange one tool for the other.

The constant exchange of instruments can have a significant impact on surgery times and negatively affect a surgeon's focus on the actual surgery. Increased surgery times invariably require greater amounts of anesthesia, which can negatively impact the patient's health and recovery. Additionally, the need for two instruments is double the cost, double the storage space, and double the effort to clean and sterilize the instruments before and after the surgery.

Accordingly, what is needed is an improved mechanism that enables a surgeon to easily operate a single device that can transition between a linearly shaped forceps tool and an angled forceps tool. However, in view of the art considered as a whole at the time the present invention was made, it was not obvious to those of ordinary skill in the field of this invention how the shortcomings of the prior art could be overcome.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

While certain aspects of conventional technologies have been discussed to facilitate disclosure of the invention, Applicants in no way disclaim these technical aspects, and it is contemplated that the claimed invention may encompass one or more of the conventional technical aspects discussed herein.

The present invention may address one or more of the problems and deficiencies of the prior art discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

### BRIEF SUMMARY OF THE INVENTION

The long-standing but heretofore unfulfilled need for an improved mechanism that enables a surgeon to easily operate a single device that can transition between a linearly shaped forceps tool and an angled forceps tool is now met by a new, useful, and nonobvious invention.

Some embodiments of the present invention include a medical instrument having a first shank and a second shank. Each shank extends between a proximal end of the medical instrument and a distal end of the instrument. In addition, the first shank and second shank are pivotably connected to each other about a hinge pin. The hinge pin includes a first end, a second end, and a central longitudinal axis extending therebetween.

The present invention further includes a hinge pin housing. The hinge pin housing includes a bore hole through which the hinge pin housing at least partially ensleeves the hinge pin. In some embodiments, the hinge pin housing is configured to move in a direction generally parallel to the central longitudinal axis of the hinge pin when the input actuator is actuated.

The housing also includes an outer surface with one or more rings extending laterally therefrom. Each ring at least partially encircles the hinge pin housing. In some embodiments, the one or more rings on the hinge pin housing are discontinuous from each other.

The present invention further includes an input actuator in operable communication with the hinge pin housing, such that actuation of the input actuator causes the hinge pin housing to move. An upper output gear is in operable communication with the one or more rings on the hinge pin housing, such that movement of the hinge pin housing causes the upper output gear to rotate. In addition, the upper output gear is configured to pivot about the central longitudinal axis of the hinge pin. A lower output gear is also in operable communication with the one or more rings on the hinge pin housing, such that movement of the hinge pin housing causes the lower output gear to rotate. Likewise, the lower output gear is configured to pivot about the central longitudinal axis of the hinge pin.

An upper output member is in operable communication with the upper output gear and a first pivoting jaw, which is located at a distal end of the first shank. As a result, rotation of the upper output gear causes the first pivoting jaw to pivot. Moreover, a lower output member is in operable communication with the lower output gear and a second pivoting jaw, which is located at a distal end of the second shank. Thus, rotation of the lower output gear causes the second pivoting jaw to pivot.

Some embodiments of the medical instrument further comprise an input member extending between the input actuator and the hinge pin housing. In some embodiments, the input member has a tapered wedge shape with an upper tapered surface in contact with the one or more rings on the hinge pin housing. Linear translation of the input member thereby causes the hinge pin housing to linearly translate about the length of the hinge pin. In some embodiments, a biasing component applies a force on the hinge pin housing to force the hinge pin housing towards the upper tapered surface of the input member.

Some embodiments of the medical instrument further comprise the input member having a plurality of teeth configured to engage a hinge pin pinion gear connected to the hinge pin. Linear translation of the input member causes the hinge pin to rotate about the central longitudinal axis of the hinge pin. Some embodiments also include the hinge pin housing threadedly engaging the hinge pin. A key is configured to prevent rotation of the hinge pin housing when the hinge pin rotates. Thus, rotation of the hinge pin causes linear translation of the hinge pin housing.

In some embodiments, the upper output gear is an upper pinion gear that is perpendicularly oriented with respect to the central longitudinal axis of the hinge pin and is adapted to engage the one or more rings on the hinge pin housing. Similarly, the lower output gear is a lower pinion gear that is perpendicularly oriented with respect to the central longitudinal axis of the hinge pin and is adapted to engage the one or more rings on the hinge pin housing. In some embodiments, the upper pinion gear includes a bevel gear configured to engage a proximal bevel gear on the upper output member, which is in the form of a first axle. The lower pinion gear also includes a bevel gear configured to engage a proximal bevel gear on the lower output member, which is in the form of a second axle. Actuation of the input actuator thereby causes rotation of the upper and lower pinion gears, which in turn causes rotation of the first and second axles.

In some embodiments, a distal bevel gear on the first axle is configured to operably engage a first jaw pivoting gear that controls rotation of the first pivoting jaw and a distal bevel gear on the second axle is configured to operably engage a second jaw pivoting gear that controls rotation of the second pivoting jaw.

In some embodiments, the upper output member is in the form of a linear rack having a plurality of teeth configured to operable engage the upper output gear and the lower output member in the form of a linear rack having a plurality of teeth configured to operable engage the lower output gear. Actuation of the input actuator causes rotation of the upper and lower pinion gears, which in turn causes linear translation of the upper and lower output members.

In some embodiments, the hinge pin, hinge pin housing, upper output gear, lower output gear, upper output member, and lower output member reside at least partially within the first shank, the second shank, or both the first and second shanks. In some embodiments, the medical instrument is a pair of forceps.

In some embodiments, the input actuator is configured to translate along the length of one of the shanks. In some embodiments, the first pivoting jaw pivots about an axis that is axially unaligned with an extent of the first shank and the second pivoting jaw pivots about an axis that is axially unaligned with an extent of the second shank.

An embodiment of the present invention includes a single input, dual output mechanism. The mechanism includes a linear actuator in mechanical communication with a linear input rack and the linear input rack is in communication with a hinge pinion gear. Thus, actuation of the linear actuator causes the rotatable hinge pinion gear to rotate.

The mechanism also includes a hinge pin secured to the hinge pinion gear such that the hinge pin rotates with the hinge pinion gear about a single rotational axis. The hinge pin further includes an outwardly projecting helical thread extending along its length. A cylindrical housing encases the hinge pin and includes a bore hole extending the length of the cylindrical housing that defines an internal surface, a thread receipt disposed on the internal surface, and a plurality of annular rings extending outwardly in a radial direction from an external surface. The thread receipt is configured to receive the helical thread on the hinge pin.

An embodiment of the mechanism further includes an upper output pinion perpendicularly oriented with respect to the cylindrical housing and adapted to engage the plurality of rings on the cylindrical housing. The output pinion also engages an upper output linear rack. The mechanism also includes a lower output pinion that is perpendicularly oriented with respect to the cylindrical housing and is adapted to engage the plurality of rings on the cylindrical housing and a lower output linear rack. Actuation of the linear actuator causes rotation of the hinge pin, which causes linear translation of the housing and thus rotation of the output pinions, and in turn, causes the distal ends of the medical device to pivot.

An embodiment includes a key and a key receipt disposed in the cylindrical housing to prevent rotation of the cylindrical housing. An embodiment further includes a rotational output in communication with at least one of the upper or lower linear output members. In an embodiment, the mechanism is housed within a pair of forceps with the hinge pin acting as the rotational pivot for the forceps.

An embodiment of the present invention includes a forceps tool having a first shank and a second shank hingedly connected to each other via a hinge pin. The hinge pin has a rotational axis, a hinge pinion gear secured thereto, and an outwardly projecting helical thread extending along a length of the hinge pin. The hinge pin and hinge pinion gear rotate as one about the rotational axis of the hinge pin.

In an embodiment, a linear actuator is in mechanical communication with a linear input rack. The linear input rack is in communication with the pinion gear, such that actuation of the linear actuator causes rotation of the hinge pinion gear and the hinge pin. Moreover, a cylindrical housing encases the hinge pin. The cylindrical housing includes a bore hole extending the length of the cylindrical housing that defines an internal surface. A thread receipt is disposed on the internal surface and is configured to receive the helical thread on the hinge pin. The housing further includes a plurality of annular rings extending outwardly in a radial direction from an external surface.

An embodiment further includes an upper output pinion gear that is perpendicularly oriented with respect to the cylindrical housing. The upper output pinion gear is adapted to engage the plurality of rings on the cylindrical housing and an upper linear output rack. Likewise, a lower output pinion is perpendicularly oriented with respect to the cylindrical housing and is adapted to engage the plurality of rings on the cylindrical housing and a lower linear output rack. Both the upper and lower linear output racks engage their respective upper and lower jaw-pivoting actuators. Each jaw pivoting actuator is configured to rotate its respective jaw. The upper jaw-pivoting actuator rotates the upper jaw out of parallel alignment with the first shank and the lower jaw-pivoting actuator rotates the lower jaw out of parallel alignment with the second shank. Thus, actuation of the linear actuator causes rotation of the upper and lower jaws.

In an embodiment, the upper and lower output pinion gears include output bevel gears. The respective output pinion gears and output bevel gears rotate as one. Each of the upper and lower output bevel gears is in operable communication with an axle via a proximal bevel gear. The distal end of the axle includes a distal bevel gear, which is in operable engagement with a jaw-pivoting bevel gear. Each jaw pivoting actuator is configured to rotate its respective jaw. The upper jaw-pivoting bevel gear rotates the upper jaw out of parallel alignment with the first shank and the lower jaw-pivoting bevel gear rotates the lower jaw out of parallel alignment with the second shank. Thus, actuation of the linear actuator causes rotation of the upper and lower jaws.

An embodiment of the present invention includes a unique hinge pin. The hinge pin includes a rotational axis and a hinge pinion gear attached. The hinge pin and hinge pinion gear rotate as one about the rotational axis of the hinge pin. In an embodiment, the hinge pinion gear is configured to engage a linear input rack connected to a linear actuator.

The hinge pin further includes an outwardly projecting helical thread extending along a length of the hinge pin. Furthermore, a cylindrical housing encases the hinge pin. The cylindrical housing has a bore hole extending the length of the cylindrical housing that defines an internal surface. The internal surface has a thread receipt disposed on the internal surface that is configured to receive the helical thread on the hinge pin. Thus, rotation of the hinge pin causes linear translation of the housing.

The cylindrical housing further includes a plurality of annular rings extending outwardly in a radial direction from an external surface. The plurality of annular rings is configured to engage and thereby cause rotation of an upper output pinion and a lower output pinion. Thus, actuation of the linear actuator causes rotation of the upper and lower pinions.

In an embodiment, each of the upper output pinion and a lower output pinion engage a linear output rack connected to a jaw pivoting actuator. In an embodiment, each of the upper output pinion and a lower output pinion are in operable communication with an axle, which engages a jaw pivoting actuator.

These and other important objects, advantages, and features of the invention will become clear as this disclosure proceeds.

The invention accordingly comprises the features of construction, combination of elements, and arrangement of parts that will be exemplified in the disclosure set forth hereinafter and the scope of the invention will be indicated in the claims.

### BRIEF DESCRIPTION OF THE DRAWINGS

For a fuller understanding of the invention, reference should be made to the following detailed description, taken in connection with the accompanying drawings, in which:

FIG. 1A is a prior art linearly shaped forceps tool.

FIG. 1B is a prior art right-angled forceps tool.

FIG. 2 is a perspective view of an embodiment of the invention in the form of a forceps tool.

FIG. 3 is a perspective view of an embodiment of the invention in the form of a forceps tool showing the distal end of the forceps pivoted into a non-linear orientation.

FIG. 4 is a perspective view of an embodiment of the novel hinge pin assembly.

FIG. 5A is a section view of FIG. 4 depicting the helical thread within the outer housing.

FIG. 5B is a perspective view of an embodiment of the outer housing.

FIG. 6 is an elevation view of an embodiment of the hinge pin.

FIG. 7 is a perspective view of an embodiment of the single input, dual output mechanism.

FIG. 8 is a top view of the mechanism from FIG. 7 without the linear input assembly. FIG. 8 highlights the key receipt in the housing.

FIG. 9 is a front view of FIG. 7.

FIG. 10 is a side view of an embodiment of the novel mechanism depicting how the input affects the outputs.

FIG. 11 is a perspective view of an embodiment of the novel single input, dual output mechanism.

FIG. 12 is a perspective view of an embodiment of the novel single input, dual output mechanism depicting how the input affects the outputs.

FIG. 13 is a perspective view of an embodiment of the mechanism within a housing.

FIG. 14 is a side elevation view of FIG. 13.

FIG. 15 is a front elevation view of FIG. 13.

FIG. 16 is a perspective view of an embodiment of the invention in the form of a forceps tool.

FIG. 17 is a view of the embodiment depicted in FIG. 16 in which the jaws of the forceps have been moved to an open position.

FIG. 18 depicts the device in FIG. 17 with the jaws having been pivoted downwards using the input actuator.

FIG. 19 depicts the device in FIG. 18 with the jaws having been moved to more of a closed position.

FIG. 20 is a side view of embodiment of the present invention.

FIG. 21 is a close-up view FIG. 20.

FIG. 22 is a perspective view of an embodiment of the present invention with the housing around the pivot pin removed to depict an embodiment of the pivot pin.

FIG. 23 is a top perspective view of FIG. 20.

FIG. 24 is a close-up view of an embodiment of the hinge pin.

FIG. 25 is a close-up view of the internal operable connection between the axle and the pivoting jaws in an embodiment of the present invention.

FIG. 26 depicts an embodiment of the present invention having skirts shown in their extended orientation.

FIG. 27 depicts the upper skirts in a retracted orientation.

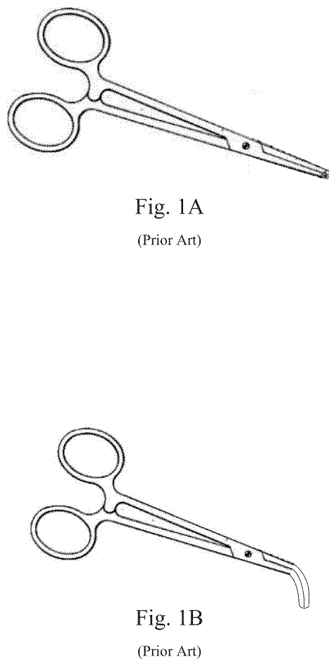

### DETAILED DESCRIPTION OF THE INVENTION

In the following detailed description of the preferred embodiments, reference is made to the accompanying drawings, which form a part thereof, and within which are shown by way of illustration specific embodiments by which the invention may be practiced. It is to be understood that other embodiments may be utilized, and structural changes may be made without departing from the scope of the invention.

As used in this specification and the appended claims, the singular forms “a,” “an,” and “the” include plural referents unless the content clearly dictates otherwise. As used in this specification and the appended claims, the term “or” is generally employed in its sense including “and/or” unless the context clearly dictates otherwise.

As used herein, “substantially perpendicular” will mean that two objects or axes are exactly or almost perpendicular, i.e., at least within five degrees or ten degrees of perpendicular, or more preferably within less than one degree of perpendicular. Similarly, the term “substantially parallel” will mean that two objects or axes are exactly or almost parallel, i.e., are at least within five or ten degrees of parallel and are preferably within less than one degree of parallel.

The present invention includes a device having a single input mechanism configured to produce a dual output. In some embodiments, the present invention includes a medical device, such as a pair of forceps. However, the device may be used outside of the medical field.

The present invention includes a hinge pin assembly. Some embodiments of the hinge pin assembly include a unique hinge pin housing that ensleeves a novel hinge pin. In some embodiments, the hinge pin assembly is designed to convert rotation of the hinge pin into linear translation of the housing along the length of the hinge pin. Some embodiments do not require rotation of the hinge pin and are configured to convert the input into linear translation of the housing along the length of the hinge pin.

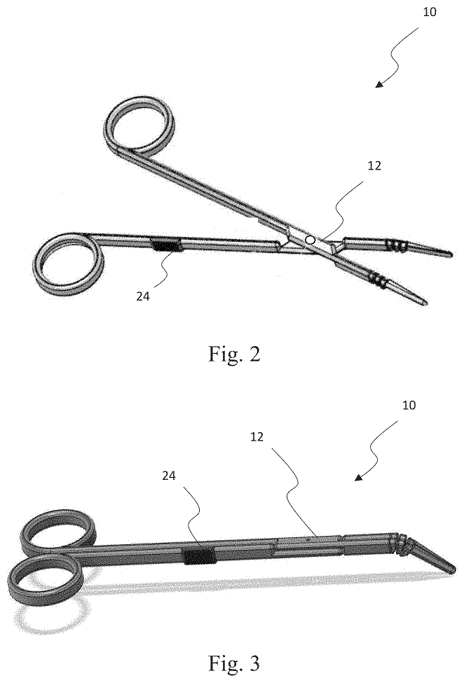

Some embodiments of the present invention include a device containing the novel hinge pin assembly residing between an input actuator and dual rotational outputs. In an embodiment the mechanism is disposed within or in connection with a medical tool such as forceps 10 as exemplified in FIGS. 2-3. Each will be discussed in more detail below.

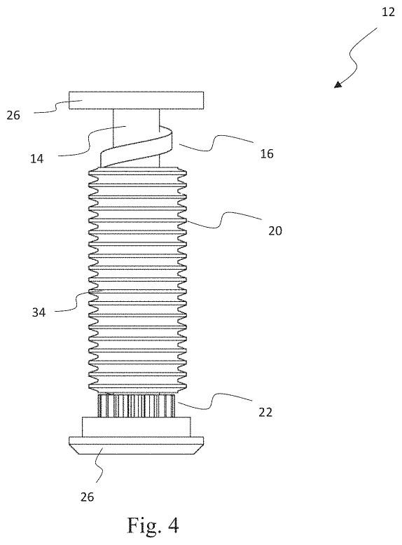

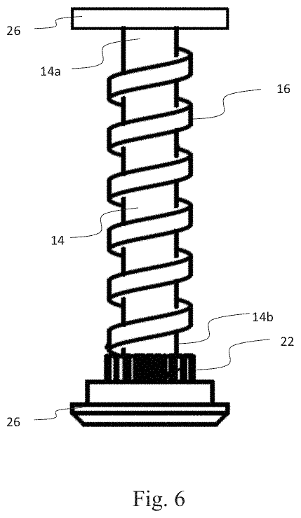

As depicted in FIGS. 4-6, an embodiment of hinge pin assembly 12 includes hinge pin 14 and a generally cylindrical-shaped hinge pin housing 20 (“housing 20”) that ensleeves hinge pin 14. Hinge pin 14 has a length extending between first end 14a and second end 14b with a central rotational axis extending along the length of hinge pin 14. An outer surface of hinge pin 14 includes helical thread 16 designed to threadedly engage thread receipt 18 on an internal surface of housing 20. In an embodiment, helical thread 16 may be disposed on the internal surface of housing 20 and thread receipt 18 is disposed on hinge pin 14.

In an embodiment, hinge pin 14 includes hinge pinion gear 22 attached thereto or integrated therewith. Hinge pinion gear 22 is rotationally fixed with respect to hinge pin 14, such that both hinge pin 14 and hinge pinion gear 22 rotate as one. In an embodiment, hinge pinion gear 22 may simply be interconnected with hinge pin 14 rather than rotationally fixed with respect to hinge pin 14; however, hinge pinion gear 22 is configured to cause rotation of hinge pin 14 when input actuator 24 is actuated. Embodiments may also use alternative types of gears or other types of force transferring components that are in mechanical, magnetic, electromagnetic and/or any other type of operable communication with hinge pin 14 to cause hinge pin 14 to rotate.

In an embodiment, hinge pinion gear 22 is connected to hinge pin 14 in a location in which hinge pinion gear 22 resides within an outer perimeter of a structural component (e.g., one or more shanks of a pair of forceps) that rotates about hinge pin 14. For example, hinge pinion gear 22 may reside proximate to the first or second ends 14a, 14b of hinge pin 14, but will remain internal with respect to the outer perimeter of the structural component to avoid incidental contact with objects such as a patient's tissue.

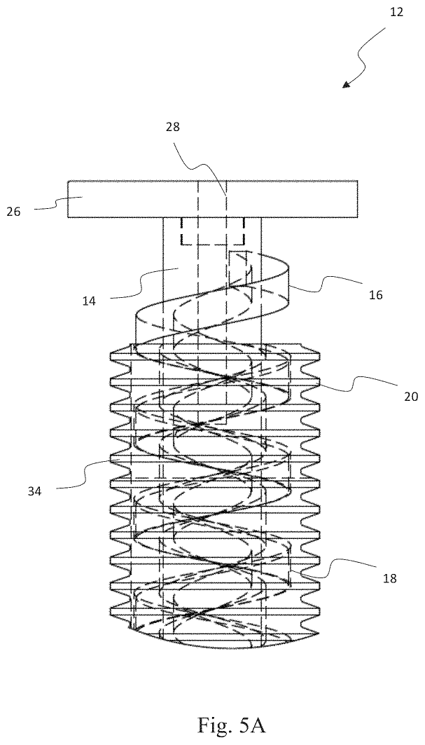

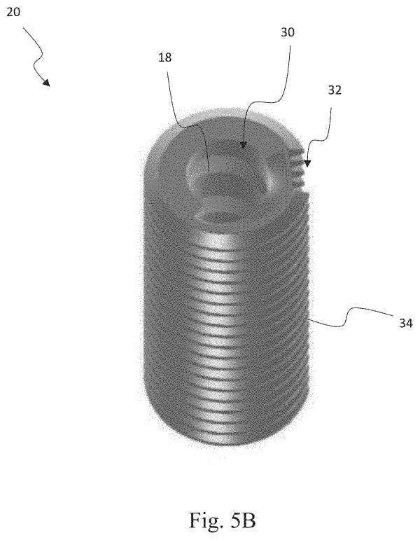

In an embodiment, the first and second ends 14a, 14b of hinge pin 14 are integrated with or attachable to retention caps 26 to secure hinge pin 14 to the structural components that rotate about hinge pin 14. As an example, retention caps 26 may be secured to hinge pin 14 via retention bolt 28 as depicted in FIG. 5A. However, alternative methods and devices can be used to secure retention caps 26 to hinge pin 14.

As best depicted in FIGS. 5, housing 20 has central bore hole 30 establishing an internal surface. Central bore hole 30 has an inner diameter that is equal to or slightly larger than the outer diameter of hinge pin 14 (excluding the helical thread). The internal surface includes thread receipt 18 configured to receive helical thread 16 on the outer surface of hinge pin 14. Additionally, housing 20 includes key receipt 32 that receives a key to prevent housing 20 from rotating about the rotational axis. Alternatively, housing 20 may have a key which engages a non-rotational key receipt. Thus, rotation of hinge pin 14 causes housing 20 to linearly translate along the length of hinge pin 14.

In an embodiment, the length of housing 20 is less than the length of hinge pin 14 or the distance between the oppositely arranged retention caps 26. Because housing 20 has a shorter length than hinge pin 14, housing 20 can linearly translate along the length of hinge pin 14.

The outer surface of housing 20 includes a plurality of longitudinally spaced annular rings 34. In an embodiment, annular rings 34 are discrete rings. Each annular ring 34 has a center axis that is concentrically aligned with the rotational axis of hinge pin 14. In an embodiment, the plurality of annular rings 34 is titled off axis with respect to the rotational axis of hinge pin 14. Moreover, an embodiment may include the plurality of annular rings 34 being a helical thread.

In some embodiments, rings 34 are semi-annular and reside at a location in which output pinion gears 40, 42 remain in contact with rings 34. In some embodiments, rings 34 are located on one or more subsections of the length of housing 20, rather than extending the entire or majority of the length of housing 20.

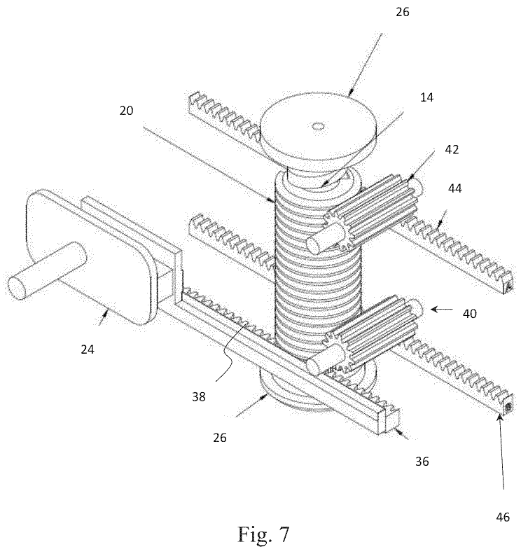

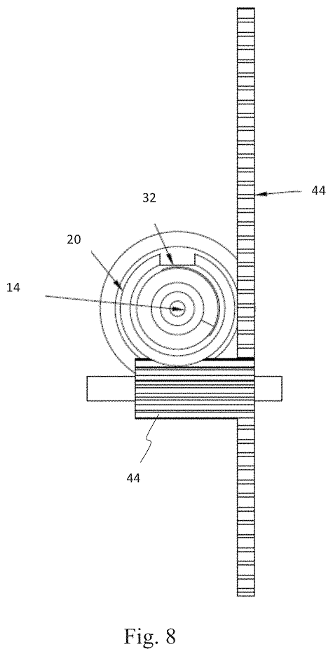

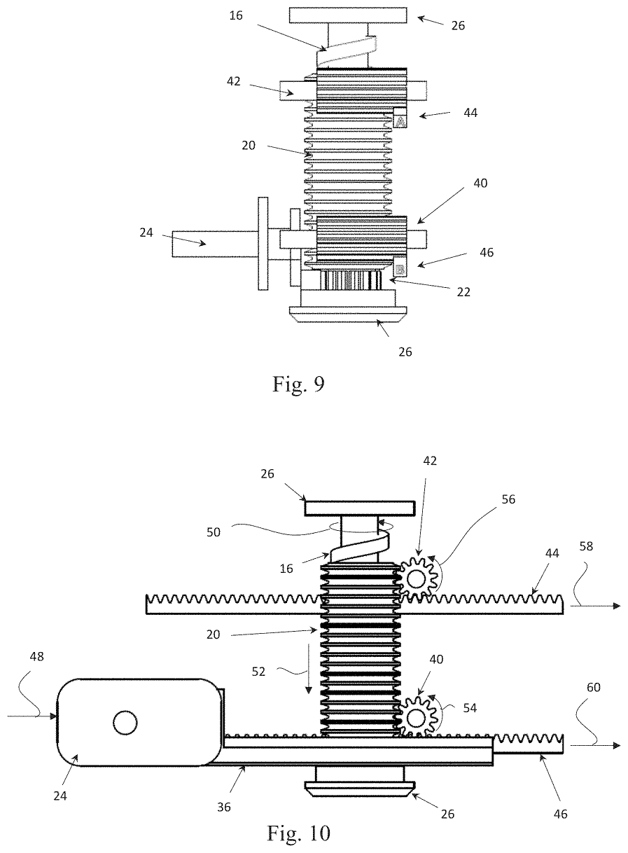

Referring now to FIGS. 7-10, the novel single input, dual output mechanism includes an input assembly that causes hinge pin 14 to rotate and an output assembly that is actuated as a result of the rotation of hinge pin 14. The input assembly includes input actuator 24 and a force transfer mechanism that mechanically interconnects the input actuator with hinge pin 14 and/or housing 20.

In an embodiment, input actuator 24 is a single linear actuator adapted to be manually actuated by a user. Input actuator 24 is attached to or integrated with the force transfer mechanism configured to operably engage hinge pin assembly 12. It should be noted that while the illustrated embodiments depict a manually controlled input actuator 24, input actuator 24 may be controlled by other devices and systems including but not limited to motorized devices, magnetic devices, and/or electromagnetic devices. In addition, while the illustrated input actuator 24 is depicted as a slide button, some embodiments may include alternative methods and devices for actuating input actuator 24.

In an embodiment, as best depicted best in FIG. 7, the force transfer mechanism is in the form of linear gear rack 36, also referred to as input rack 36. Input rack 36 includes a plurality of teeth 38 on at least one surface. Teeth 38 of input rack 36 are sized and shaped to engage hinge pinion gear 22. Thus, as best depicted in FIG. 10, linear translation of input actuator 24 via an input force as represented by arrow 48 causes linear translation of input rack 36. Input rack 36 causes rotation (as represented by arrow 50) of hinge pin 14 through the operable communication of input rack 36 and hinge pinion gear 22. The rotation of hinge pin 14 causes housing 20 to linearly translate, as represented by arrow 52, on account of its threaded engagement with hinge pin 14.

The mechanism further includes an output assembly. The output assembly is in operable communication with the input assembly, such that actuation of input 24 causes one or more output reactions. In an embodiment, the output assembly includes as least one output pinion gear and at least one output member. However, some embodiments include at least two output pinion gears and at least two output members.

As best depicted in FIGS. 7 and 10, an embodiment of the output assembly includes upper output pinion gear 40 and lower output pinion gear 42. Both are in mechanical, magnetic, electromagnetic and/or any other type of operable communication with housing 20. As best depicted in FIG. 10, the depicted embodiment includes output pinion gears 40, 42 configured to rotate as represented by arrows 54, 56 when housing 20 translates linearly in accordance with arrow 52.

Upper output pinion 42 also engages an upper output member in the form of output rack 44. Likewise, lower output pinion 40 engages a lower output member in the form of lower output rack 46. Thus, the translation of housing 20, caused by rotation of hinge pin 14 from the input assembly, causes both upper and lower output pinions 40, 42 to rotate, which in turn causes, upper and lower output racks 44, 46 to linearly translate as illustrated by arrows 58, 60. In other words, a single input causes dual outputs. An embodiment, however, may include the mechanism producing a single output or more than two outputs.

In an embodiment, output pinions 40, 42 are connected to housing 20 in locations in which output pinions 40, 42 reside within an outer perimeter of one or more structural components (e.g., forceps shanks) that rotate about hinge pin 14. As a result, output pinions 40, 42 are shielded from unintentionally engaging external objects, such as a patient's tissue.

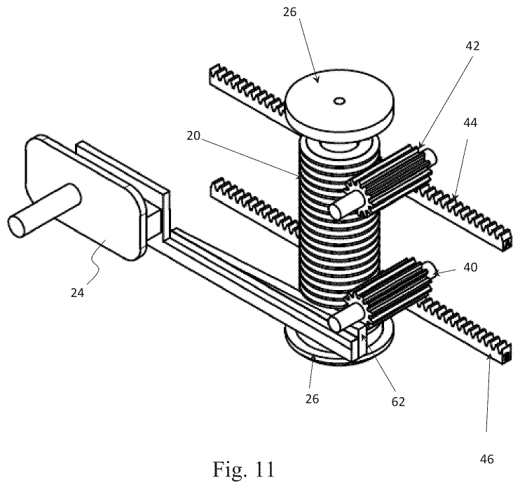

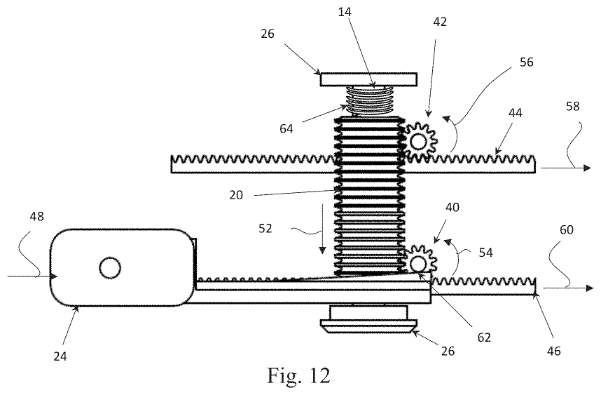

Referring now to FIGS. 11-12, an embodiment of the novel single input, dual output mechanism includes an input assembly that causes housing 20 to linearly translate and an output assembly that is actuated as a result of the translation of housing 20. The input assembly includes input actuator 24 and a force transfer mechanism that operably interconnects input actuator 24 with housing 20.

As depicted, the exemplary force transfer mechanism is in the form of tapered wedge 62. In an embodiment, tapered wedge 62 has a distal end with a greater height than the proximal end. In some embodiments, the taper is reversed so that the proximal end has a greater height than the distal end.

As shown in FIG. 10, linear translation of input actuator 24 via an input force as represented by arrow 48 causes linear translation of tapered wedge 62 as represented by arrow 52. Tapered wedge 62 causes housing 20 to linearly translate on account of at least one annular ring 34 resting on the top surface of wedge 62. In some embodiments at least some of the annular rings proximate wedge 62 are in the form of a continuous thread.

In embodiments employing the wedge shaped force transferring mechanism, housing 20 can be used without internal thread receipts. Likewise, hinge pin 14 does not require threads to allow housing 20 to translate about the length of hinge pin 14. Housing 20 can simply translate along the smooth outer surface of hinge pin 14. As a result, this embodiment is easier and cheaper to produce.

In some embodiments, as depicted in FIG. 12, a biasing component, such as spring 64, biases housing 20 towards wedge 62 to ensure that housing 20 is constantly in operable communication with wedge 62 even if the mechanism is inverted. The biasing component may be any biasing device known to a person of ordinary skill in the art including but not limited to springs, electromagnets, and permanent magnetics.

Similar to other embodiments, the output assembly is in operable communication with the input assembly, such that actuation of input 24 causes one or more output reactions. As best depicted in FIG. 12, an embodiment of the output assembly includes upper output pinion gear 40 and lower output pinion gear 42. Both are in mechanical, magnetic, electromagnetic and/or any other type of operable communication with housing 20. As best depicted in FIG. 10, the depicted embodiment includes output pinion gears 40, 42 configured to rotate as represented by arrows 54, 56 when housing 20 translates linearly in accordance with arrow 52.

Upper output pinion 42 also engages upper output rack 44. Likewise, lower output pinion 40 engages lower output rack 46. Thus, the translation of housing 20 caused by wedge 62 results in both upper and lower output pinions 40, 42 rotating, which in turn causes, upper and lower output racks 44, 46 to linearly translate as illustrated by arrows 58, 60. In other words, a single input causes dual outputs. An embodiment, however, may include the mechanism producing a single output or more than two outputs.

In some embodiments, one or more of the output racks 44, 46 are in mechanical communication with a rotational output mechanism, such as a pinion gear. In some embodiments, the rotational output mechanism can convert a single linear input into one or more rotational outputs.

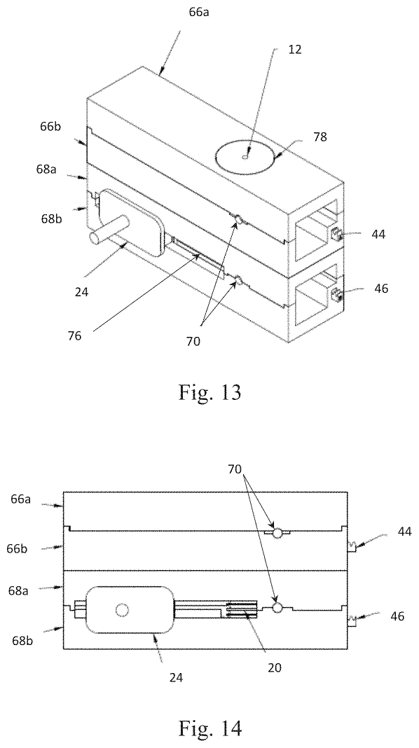

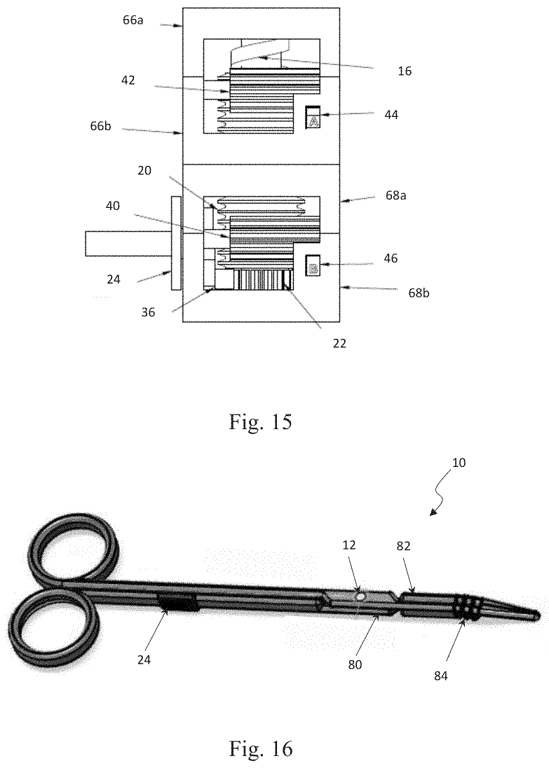

In an embodiment as depicted in FIGS. 13-15, upper output pinion 42 and upper output rack 44 reside within first structural member 66 while lower output pinion 40 and lower output rack 46 reside within second structural member 68. Both the first and second structural members 66, 68, have pinion retention receipts 70 that respectively retain upper and lower output pinions 40, 42. Both structural members 66, 68 also include output rack channels 72, 74 and at least one of the structural members includes an actuator channel 76. In an embodiment, both structural members 66, 68 are respectively comprised of upper sections 66a, 68a and lower sections 66b, 68b that form structural members 66, 68 when assembled.

First and second structural members 66, 68 also include hinge receipt 78 that houses hinge pin assembly 12. As a result, first and second structural members 66, 68 are hingedly secured to each other about hinge pin 14 thereby allowing first and second structural members 66, 68 to be rotated with respect to each other about hinge pin 14. Structural members 66, 68 also carry upper and lower output pinions 40, 42 in a pivotable/rotational manner about the circumference of housing 20 while remaining in mechanical communication with annular rings 34. Thus, input actuator 24 will cause upper and lower output pinions 40, 42 to rotate about their respective rotational axes regardless of whether first and second structural members 66, 68 have been rotated with respect to each other.

It should be noted that FIGS. 13-15 depict the embodiment of the input assembly and hinge assembly 12 as depicted in FIGS. 7-10, however, first and second structural members 66, 68 may house the input assembly and hinge assembly 12 as depicted in FIGS. 11-12.

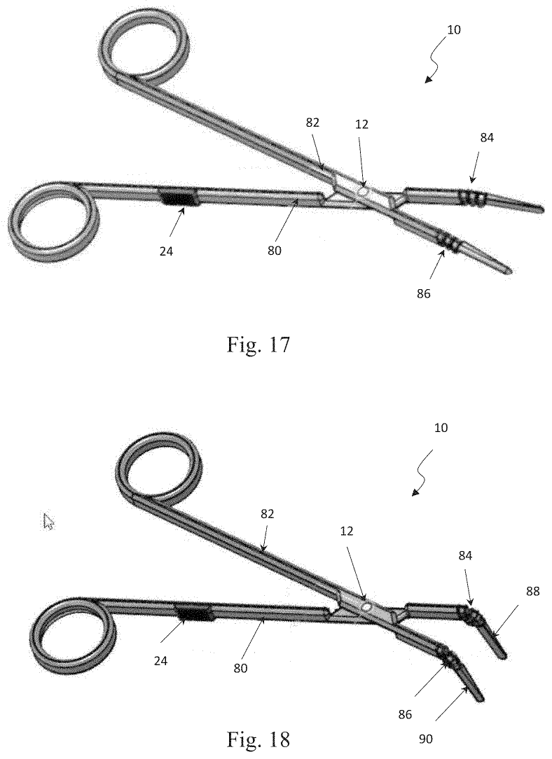

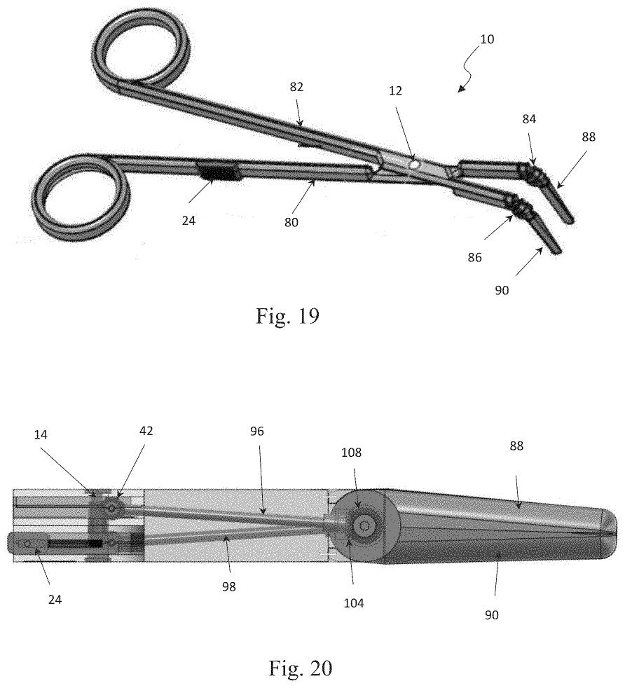

Referring now to FIGS. 16-19, an embodiment of the present invention includes the single input, dual output mechanism described above residing within a medical tool, such as the depicted pair of forceps 10. Hinge pin assembly 12 resides at a typical hinge location for a pair of forceps and each shank 80, 82 of the forceps houses one of output pinions 40, 42 and output racks 44, 46. As depicted, input actuator 24 is disposed in lower shank 80 and can translate linearly along the length of lower shank 80. Upper shank 82 houses the upper output pinion 42 and upper output rack 44, while lower shank 80 houses lower output pinion 40 and lower output rack 46. The outputs respond to input from input actuator 24 in the same or similar manner as described above.

The embodiment of the invention depicted in FIGS. 16-19 further includes pivot mechanism(s) disposed proximate to the distal ends of the forceps to allow the distal ends to pivot out of a linear orientation with respect to the shanks. In an embodiment, both lower and upper linear output racks 46, 44 separately and respectively engage lower and upper jaw-pivoting actuators 84, 86. Each jaw pivoting actuator 84, 86 is configured to rotate its respective jaw 88, 90. Upper jaw-pivoting actuator 86 rotates first jaw 90 out of parallel alignment with upper shank 82 and lower jaw-pivoting actuator 84 rotates second jaw 88 out of parallel alignment with lower shank 80. Thus, actuation of input actuator 24 causes rotation of jaws 88, 90.

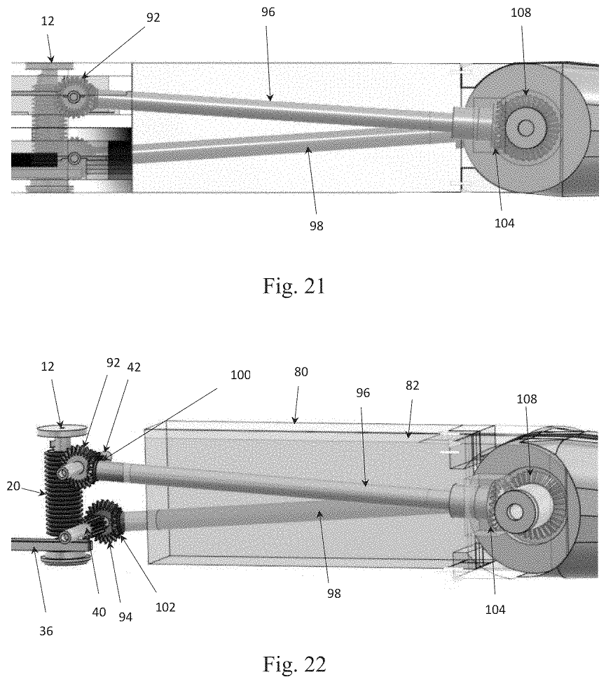

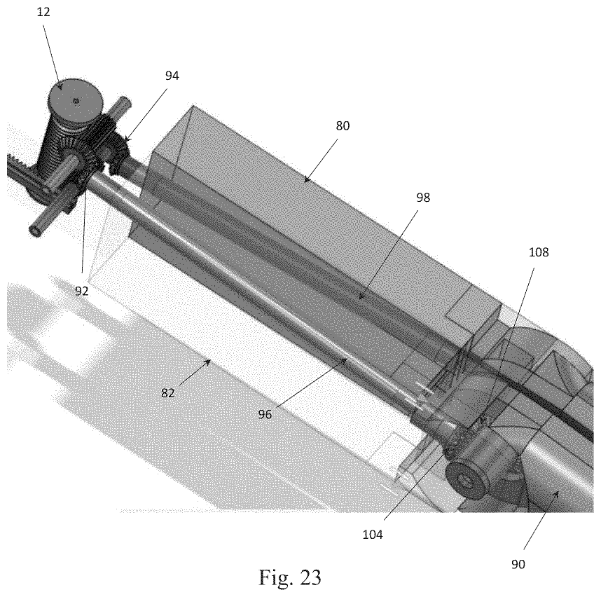

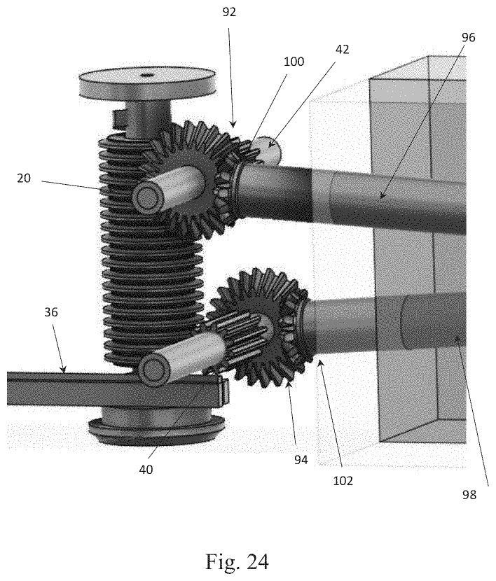

Some embodiments of the output assembly, as best depicted in FIGS. 20-27, include the output members in the form of first/upper axle 96 and second/lower axle 98. Each axle 96, 98 respectively includes proximal bevel gears 100, 102 and distal bevel gears 104, 106.

These embodiments further include upper output pinion gear 42 in operable communication with upper output bevel gear 92 and lower output pinion gear 40 in operable communication lower output bevel gear 94. In an embodiment, the respective output pinion gears 40, 42 and output bevel gears 92, 94 rotate as one.

Upper axle 96 is in operable engagement with upper output bevel gear 92 via proximal bevel gear 100. Lower axle 98 is in operable engagement with lower output bevel gear 94 via proximal bevel gear 102. Thus, rotation of output pinion gears 40, 42 cause rotation of output bevel gears 92, 94, which in turn rotate axles 96, 98.

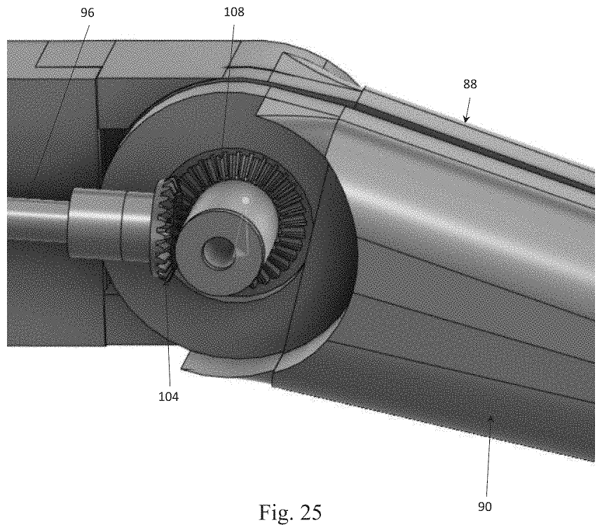

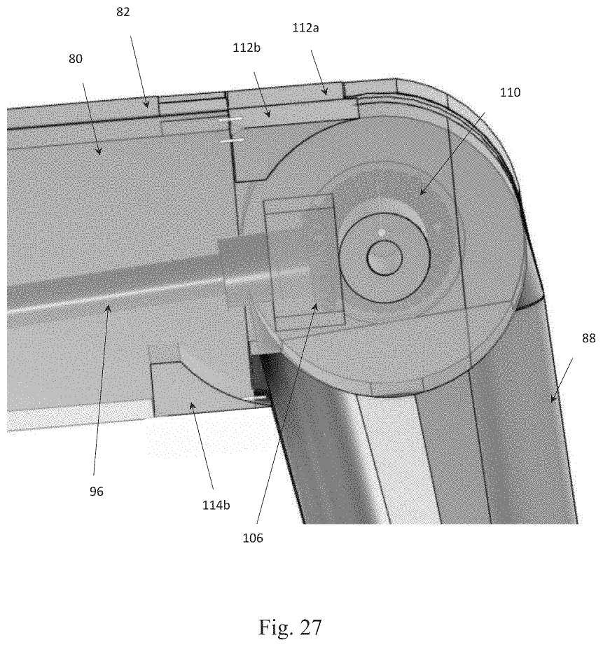

The distal ends of each axle include a distal bevel gear. Upper axle 96 includes distal bevel gear 104. Lower axle 98 includes distal bevel gear 106. Distal bevel gear 104 of upper axle 96 engages first jaw-pivoting bevel gear 108 and distal bevel gear 106 of lower axle 98 engages second lower jaw-pivoting bevel gear 110 oppositely arranged from the first. In viewing FIG. 25, first jaw-pivoting bevel gear 108 has a center rotational axis generally extending out of the paper in a generally perpendicular manner. Second lower jaw-pivoting bevel gear 110 is oppositely arranged such that the center rotational axis of second jaw-pivoting bevel gear 110 extends into the paper in a generally perpendicular manner. Second lower jaw-pivoting bevel gear 110 and distal bevel gear 106 can be seen in FIG. 27. Each jaw pivoting bevel gear 108, 110 is operably configured to rotate its respective jaw 90, 88.

In operation, rotation of upper output pinion 40 causes rotation of upper output bevel gear 92. Rotation of upper output bevel gear 92 causes rotation of proximal bevel gear 100 of upper axle 96, which in turn rotates upper axle 96. Rotation of upper axle 96 causes distal bevel gear 104 to rotate. Rotation of distal bevel gear 104 causes rotation of first jaw-pivoting bevel gear 108, which ultimately causes first jaw 90 to pivot about its pivotal axis.

Likewise, rotation of lower output pinion 40 causes rotation of lower output bevel gear 94. Rotation of lower output bevel gear 94 causes proximal bevel gear 102 of lower axle 98 to rotate, which in turn rotates lower axle 98. Rotation of lower axle 98 causes distal bevel gear 106 to rotate. Rotation of distal bevel gear 106 causes rotation of second jaw-pivoting bevel gear 110, which ultimately causes second jaw 88 to pivot about its axis of rotation.

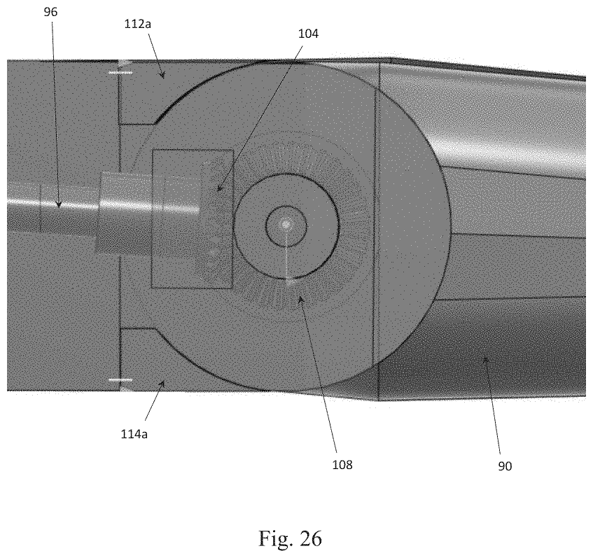

As best depicted in FIGS. 26 and 27, an embodiment includes a pair of upper skirts 112a, 112b and a pair of lower skirts 114a, 114b. The skirts are subject to a constant bias force applied in a distal direction. The skirts are designed to prevent objects from becoming lodged between shanks 80, 82 and the rotational jaws 88, 90. FIG. 27 illustrates how the skirts move when the jaws rotate. As illustrated therein, jaw 88 rotates out of longitudinal alignment with shank 80 and jaw 88 forces skirt 114b to retract in a proximal direction.

As depicted, the jaws are permitted to rotate roughly 180 degrees based on the square distal end of the shanks. An embodiment of the shanks, however, includes a tapered distal end allowing the jaws to rotate substantially beyond 180 degrees.

The advantages set forth above, and those made apparent from the foregoing description, are efficiently attained. Since certain changes may be made in the above construction without departing from the scope of the invention, it is intended that all matters contained in the foregoing description or shown in the accompanying drawings shall be interpreted as illustrative and not in a limiting sense.

It is also to be understood that the following claims are intended to cover all of the generic and specific features of the invention herein described, and all statements of the scope of the invention that, as a matter of language, might be said to fall therebetween.

What is claimed is:

1. A medical instrument, comprising:
a first shank and a second shank, each extending between a proximal end of the medical instrument and a distal end of the instrument, wherein the first shank and second shank are pivotably connected to each other about a hinge pin;
the hinge pin including a first end, a second end, and a central longitudinal axis extending therebetween;
a hinge pin housing, the hinge pin housing including:
a bore hole through which the hinge pin housing at least partially ensleeves the hinge pin;
an outer surface with one or more rings extending laterally therefrom, wherein each ring at least partially encircles the hinge pin housing;
an input actuator in operable communication with the hinge pin housing, such that actuation of the input actuator causes the hinge pin housing to move;
an upper output gear, the upper output gear:
in operable communication with the one or more rings on the hinge pin housing, such that movement of the hinge pin housing causes the upper output gear to rotate;
configured to rotate about the central longitudinal axis of the hinge pin;
a lower output gear, the lower output gear:
in operable communication with the one or more rings on the hinge pin housing, such that movement of the hinge pin housing causes the lower output gear to rotate; and
configured to rotate about the central longitudinal axis of the hinge pin;
an upper output member, the upper output member:
in operable communication with the upper output gear;
in operable communication with a first pivoting jaw located at a distal end of the first shank;
whereby rotation of the upper output gear causes the first pivoting jaw to pivot;
a lower output member, the lower output member:
in operable communication with the lower output gear;
in operable communication with a second pivoting jaw located at a distal end of the second shank; and
whereby rotation of the lower output gear causes the second pivoting jaw to pivot.

2. The medical instrument of claim 1, wherein the hinge pin housing is configured to move in a direction generally parallel to the central longitudinal axis of the hinge pin when the input actuator is actuated.

3. The medical instrument of claim 1, further comprising:
an input member extending between the input actuator and the hinge pin housing;
the input member having a tapered wedge shape with an upper tapered surface in contact with the one or more rings on the hinge pin housing;
whereby linear translation of the input member causes the hinge pin housing to linearly translate about the length of the hinge pin.

4. The medical instrument of claim 1, further comprising a biasing component applying a force on the hinge pin housing to force the hinge pin housing towards the upper tapered surface of the input member.

5. The medical instrument of claim 1, further comprising:
an input member extending between the input actuator and the hinge pin housing;
the input member having a plurality of teeth configured to engage a hinge pin pinion gear connected to the hinge pin;
whereby linear translation of the input member causes the hinge pin to rotate about the central longitudinal axis of the hinge pin.

6. The medical instrument of claim 5, further comprising:
the hinge pin housing threadedly engaging the hinge pin;
a key configured to prevent rotation of the hinge pin housing when the hinge pin rotates;
whereby rotation of the hinge pin causes linear translation of the hinge pin housing.

7. The medical instrument of claim 1, wherein the one or more rings on the hinge pin housing are discontinuous from each other.

8. The medical instrument of claim 1, wherein:
the upper output gear is an upper pinion gear that is perpendicularly oriented with respect to the central longitudinal axis of the hinge pin and is adapted to engage the one or more rings on the hinge pin housing; and
the lower output gear is a lower pinion gear that is perpendicularly oriented with respect to the central longitudinal axis of the hinge pin and is adapted to engage the one or more rings on the hinge pin housing.

9. The medical instrument of claim 8, further including:
the upper pinion gear including a bevel gear configured to engage a proximal bevel gear on the upper output member, which is in the form of a first axle;
the lower pinion gear including a bevel gear configured to engage a proximal bevel gear on the lower output member, which is in the form of a second axle;
whereby actuation of the input actuator causes rotation of the upper and lower pinion gears, which in turn causes rotation of the first and second axles.

10. The medical instrument of claim 9, further including:
a distal bevel gear on the first axle configured to operably engage a first jaw pivoting gear that controls rotation of the first pivoting jaw; and
a distal bevel gear on the second axle configured to operably engage a second jaw pivoting gear that controls rotation of the second pivoting jaw.

11. The medical instrument of claim 8, further including:
the upper output member in the form of a linear rack having a plurality of teeth configured to operable engage the upper output gear;
the lower output member in the form of a linear rack having a plurality of teeth configured to operable engage the lower output gear;
whereby actuation of the input actuator causes rotation of the upper and lower pinion gears, which in turn causes linear translation of the upper and lower output members.

12. The medical instrument of claim 1, wherein the hinge pin, hinge pin housing, upper output gear, lower output gear, upper output member, and lower output member reside at least partially within the first shank, the second shank, or both the first and second shanks.

13. The medical instrument of claim 1, wherein the input actuator is configured to translate along the length of one of the shanks.

14. The medical instrument of claim 1, wherein the medical instrument is a pair of forceps.

15. The medical instrument of claim 1, wherein the first pivoting jaw pivots about an axis that is axially unaligned with an extent of the first shank and the second pivoting jaw pivots about an axis that is axially unaligned with an extent of the second shank.
